(19) United States
(12) Patent Application Publication
(10) Pub. No.: US 2011/0129889 A1
(43) Pub. Date: Jun. 2, 2011

(54) Process for Production of Ethanol from Lignocellulosic Material

(75) Inventors: Shashank Narayan Inamdar, Maharashtra (IN); Venkata Ramakrishna Sonti, Maharashtra (IN); Ravikumar Rao, Maharashtra (IN); Milind Shrikant Kulkarni, Maharashtra (IN); Prasad Rao, Maharashtra (IN); Mohan Babu, Maharashtra (IN); Satyendra Waman Joshi, Maharashtra (IN); Anilkumar Subhash Lokare, Maharashtra (IN); Prashant Prakash Gokhale, Maharashtra (IN); Ashvini Monish Shete, Maharashtra (IN); Gunjan Nabakumar Mukharjee, Maharashtra (IN)

(73) Assignee: PRAJ INDUSTRIES LIMITED, Maharashtra (IN)

(21) Appl. No.: 13/055,119

(22) PCT Filed: Jul. 20, 2009
(86) PCT No.: PCT/IN09/00410

§ 371 Date: Jan. 20, 2011

(30) Foreign Application Priority Data
Jul. 21, 2008 (IN) 878/MUM/2008
Jul. 20, 2009 (IN) PCT/IN2009/000410

(51) Int. Cl. C12P 7/10

(52) U.S. Cl. 435/165

(57) ABSTRACT

This invention relates to a process for production of ethanol from lignocellulosic material. Lignocellulosic material treated with a dicarboxylic acid, preferably with oxalic acid, separating a hemicellulosic fraction to ferment pentose sugar. The lignin is dissolved in alkali, preferably with NaOH, separating a cellulose fraction for further enzymatic treatment with one or more than one cellulytic enzymes capable of hydrolyzing cellulose. The enzyme hydrolyasate is further subjected to fermentation in the presence of ethanol-producing yeast, preferably Sacchoromyces cerevisiae. The fermented broth is further subjected to distillation followed by dehydration to yield ethanol.

### CROSS-REFERENCE TO RELATED APPLICATION

This application claims priority from patent application filed on Jul. 21, 2008 at the Indian patent office having patent application no. 878/MUM/2008.

### FIELD OF THE INVENTION

The present invention relates to a process for production of ethanol from lignocellulosic material comprising cellulose, lignin and hemicellulose as main components. The cellulosic fraction hydrolyzed into sugars and fermented to produce ethanol.

### BACKGROUND OF THE INVENTION

The present invention relates to a process for the production of ethanol from lignocellulose-containing material. Ethanol is a well-known compound which has a wide use. Ethanol has attracted interest as an alternative liquid fuel. If the ethanol production process only uses energy from renewable energy sources, no net carbon dioxide is added to the atmosphere, making ethanol an environmentally beneficial energy source.

The conventional method of production of ethanol has been by the fermentation of sugars. All beverage ethanol and most of the industrial ethanol as well as fuel ethanol are made even today by this process. A type of raw materials which is used for ethanol production by fermentation is sugar-bearing substances such as molasses—a by-product of sugar industry, sugar cane juice, sugar beet juice that can be directly fermented. Another type of raw materials used for ethanol production by fermentation is starch-bearing substances which include grains such as sorghum, rice, wheat, barley, corn, tubers such as cassaya or potato. These starches are polymers of hexose sugars and need to be broken down to simple sugars either by enzymatic or chemical hydrolysis. Third type of raw materials used for ethanol production by fermentation is cellulosic materials which include lignocellulosic substances like straw, corncobs, wood wastes, bagasse and waste paper.

Lignocellulosic materials are composed of mainly cellulose, hemicellulose, and lignin. Cellulose is a linear, crystalline polymer of .beta.-D-glucose units. The structure is rigid and harsh treatment is usually required to break down cellulose. Hemicellulose has usually as a main component linear and branched heteropolymers of L-arabinose, D-galactose, D-glucose, D-mannose, D-xylose and L-rhamnose. The composition of hemicellulose varies with the origin of the lignocellulosic material. The structure is not at least totally crystalline and is therefore usually easier to hydrolyze than cellulose. Examples of lignocellulosic materials considered for ethanol production are hardwood, softwood, forestry residues, agricultural residues, and municipal solid waste (MSW). Both cellulose and hemicellulose can be used for ethanol production. The pentose content in the raw material is of importance as pentoses are often difficult to ferment to ethanol. The pentose content can comprise 6-28% of the total dry matter. To achieve maximum ethanol yield, all monosaccharide should be fermented. Softwood hemicellulose contains a high proportion of mannose and more galactose and glucose than hardwood hemicellulose whereas hardwood hemicellulose usually contains a higher proportion of pentoses like D-xylose and L-arabinose.

The composition of the main components varies depending on the type of biomass. Typical composition on dry basis by weight is as follows:

| Lignocellulosic | | | |
|---|---|---|---|
| Feed Stock | Cellulose | Hemi cellulose | Lignin |
| Corn Stover | 22-29 | 13-18 | 12-17 |
| Corn Cob | 25-35 | 25-32 | 13-17 |
| Grasses | 25-34 | 12-16 | 18-22 |
| Soft woods | 35 | 20-25 | 22-25 |
| Corn DWG | 17 | 14-18 | 22-25 |
| Cane bagasse | 38-45 | 18-25 | 22-25 |

Lignocellulose represents a very cheap and readily available substrate for the preparation of sugars which may be used alone or microbial fermented to produce alcohols and other industrial chemicals. The technology for production of ethanol from grain, sugar based feed stocks like molasses, sugar cane syrup, sugar beet and fruit for beverage purposes has been well developed for centuries. However, the costs have been relatively high compared to the cost of gasoline. Accordingly, many methods have been proposed to reduce the cost and increase the efficiency of ethanol production.

Ethanol production from lignocellulosic material can comprise the following steps: (1) degradation of the lignocellulosic structure to a fermentable substrate, (2) fermentation of the fermentable substrate, and (3) distillation of the fermentation broth to obtain ethanol. In the past, there have been problems encountered in the efficient conversion of the lignocellulosic hydrolysates to ethanol. First, after pretreatment, the hydrolysate contains not only fermentable sugars, but also a broad range of compounds which often have inhibitory effects in the microorganisms used for fermentation. The composition of these compounds depends upon the type of lignocellulosic material used and the chemistry and nature of the pretreatment process. Second, the hemicellulose hydrolysates contain not only hexoses but also pentoses. The pentose fraction in hemicellulose comprises mainly xylose, but depending on the raw material origin, the arabinose fraction may be substantial. While some hexoses can readily be fermented, pentoses are more difficult to ferment. There are several methods known in the art which use different acids for pre-hydrolysis or enzymatic hydrolysis.

The prolonged acid pretreatment to lignocellulosic material leads to degradation of glucose derived from cellulose into hydroxymethylfurfural which can be further degraded into levulinic acid and formic acid. Xylose, which is formed from hemicellulose, is degraded by acids into furfural and then results in tars and other degradation products. The sugar degradation not only reduces the sugar yield, but the furfural and other by-products can inhibit the fermentation process. Thus, though the dilute acid process has the advantage of faster reaction, yet it has the biggest drawback of low sugar yield.

Another drawback of using inorganic acid is that for complete hydrolyzes by acid of the cellulose and hemicellulose in a lignocellulosic substrate, degradation of the desirable sugars and formation of the toxic byproducts cannot be avoided due to kinetic constraints. In case sufficiently gentle conditions are used so that only an insignificant degradation of sugars occurs, then in that case it does not result in complete hydrolysis of substrate. Further drawback of using inorganic acids is that, they are corrosive and require special handling means and equipment.

In order to avoid the above drawbacks of using inorganic acid, the pretreatment is known to be carried out by enzymatic hydrolysis of cellulose with cellulase instead of chemical pretreatment. A limitation of the above process is the availability of enzymes in bulk form and also the cost at which the enzyme are sold. Enzymatic hydrolysis may also have other imitations like substrate (Cellulose) and product (Glucose and Ethanol in case of simultaneous saccharification and fermentation).

These prior processes are all multi-step processes which are relatively costly and often have inadequate efficiency. It is, therefore, desirable to provide an improved process to produce ethanol, from lignocellulosic material in biomass.

### SUMMARY OF THE INVENTION

An improved process provided to produce ethanol from lignocellulosic material comprising of Cellulose, Hemicellulose and Lignin. Advantageously, the process is effective and economical.

In the process, the lignocellulosic material comprising cellulose, hemicellulose and lignin is subjected to size reduction, reducing it to a finely divided form. Subjecting the finely divided reduced material for slurry preparation with water or evaporated condensate or recycled process stream, reacting the said aqueous slurry with organic acid such as dicarboxylic acid specifically oxalic acid, in combination with heating, for more effective pretreatment.

The process further includes separating the solids and hemicellulose rich supernatant from the pretreated slurry of Lignocellulosic material and subjecting the hemicellulose rich fraction for pentose fermentation by a yeast preferably Pichia stipits. The solid residue containing cellulose and lignin is subjected to slurry preparation and treated with alkali in order to remove lignin and separating out the delignified cellulose rich solid wet cake.

The process further includes washing the cellulose rich solid wet cake for further enzymatic treatment containing at least one enzyme capable of digesting cellulose contained in the lignocellulosic material into glucose. The enzyme hydrolysate further submitted to fermentation, to produce ethanol, in the presence of a yeast strain Saccharomyces cerevisiae suitably acclimatized to the principle substrates in the hydrolysate to yield ethanol. The resultant ethanol further separated by distillation and processed by dehydration to achieve effective purity.

A more detail explanation of the invention is provided in to following description and appended claims taken in conjunction with the accompanying drawing.

### BRIEF DESCRIPTION OF THE DRAWING

FIG. 1 illustrates the entire process flow chart for the production of ethanol from the lignocellulosic material. The process of the present invention is illustrated with a flow chart which is intended to illustrate the steps of the process and is not intended to be taken restrictively to imply any limitation on the scope of the present invention in the accompanying figure.

### DETAILED DESCRIPTION OF THE INVENTION

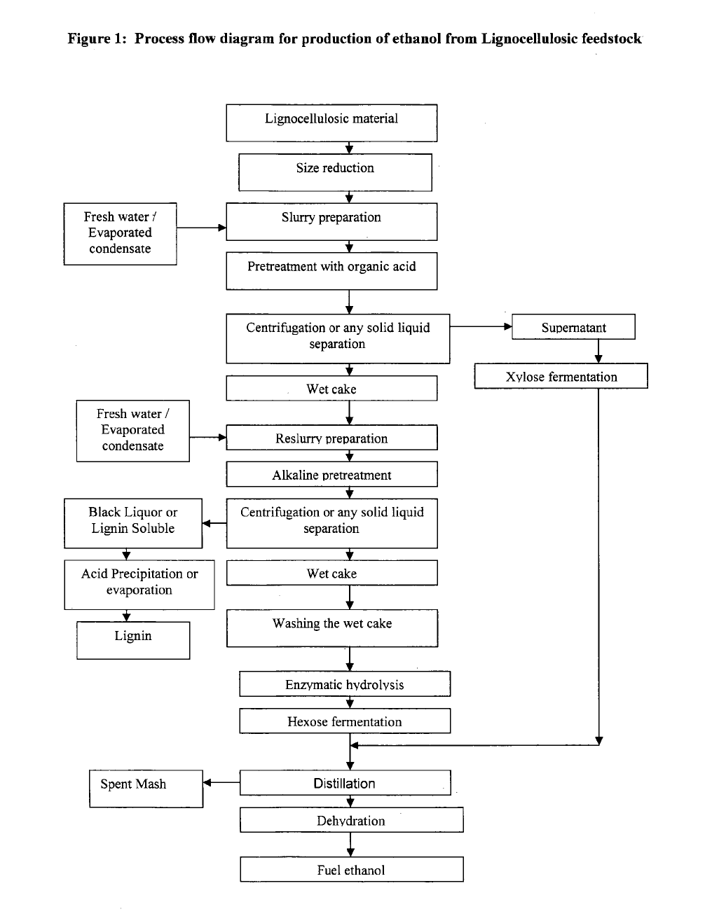

The following is a detail description and explanation of the preferred embodiments of the process of the invention with some examples thereof. In order to better appreciate the invention, it is described with reference to the process flow chart in FIG. 1 which illustrates the preferred embodiment of the invention.

As indicated in FIG. 1, lignocellulosic material is initially subjected to size reduction using any suitable mechanical action to reducing it to finely divided form. This finely divided lignocellulosic material is mixed with water or evaporated condensate or recycled process stream to produce the aqueous slurry, which is further pretreated by reacting it with organic acid such as dicarboxylic acid, preferably oxalic acid in combination with heating. The organic acid pretreated slurry is subjected to filtration or centrifugation for separating the supernatant containing hemicellulose and solids containing lignin and cellulose. The oxalic acid treatment is advantageous over other acid treatment by having minimum cellulose loss. The supernatant further processed for at least one pentose sugar fermentation preferably xylose by a yeast preferably Pichia stipits. The solids containing lignin and cellulose further mixed with water or evaporated condensate or recycled process stream to produce the aqueous slurry for receiving the alkali treatment. The alkali reacted slurry solubilize the lignin and remaining wet solid cake contains the cellulose rich fraction. The solid wet cake of cellulose is washed and treated with at least one cellulytic enzyme capable of hydrolyzing cellulose. The enzyme hydrolysate media further subjected to fermentation with addition of ethanol producing microorganism, preferably an ethanol producing yeast. The ethanol produced in fermentation is separated by distillation and further purified by dehydration.

Sugar cane bagasse, grasses, Distillers Wet Grains (DWG), Distillers Dry Grains Solubles (DDGS), corn cob and corn stover feedstock were used as representative lignocellulosic material. The lignocellulosic material is ground before pretreatment. The particle size is not critical but generally reduced particle size enables exposure of more surface area for cellulase to attach and degrade the cellulose after hydrolysis. Appropriate particle size varies with feedstock and its inherent physical properties. In the present invention, the lignocellulosic material is reduced a particle size of about 0.5 mm to about 20 mm.

Exemplary embodiments of the present invention comprises of:

### Lignocellulose Feedstock Size Reduction and Preparation of Slurry

The lignocellulosic feedstock taken has moisture content in the range of about 2% to about 10%. The feedstock is subjected to mechanical action for size reduction to about 2 mm to about 20 mm and slurry is prepared with fresh water or the recycled condensate from the evaporation step, taking solids in the range of about 30% to about 95% depending upon the feedstock. In one embodiment of the invention the grass and DWG, the solids taken in the range of about 30% to about 50%.

### Organic Acid Hydrolysis

The aqueous slurry is treated with water containing about 0.1% to about 3.0% of dicarboxylic acid preferably oxalic acid by weight of slurry wherein the slurry is taken at a solid concentration varying about 7% to about 25% by weight preferably about 5% to about 15%. The slurry is hydrolyzed at temperature of about 110° C. to about 210° C. preferably at temperature in the range of about 130° C. to about 200° C. more preferably in the range of about 140° C. to about 170° C. for a period of about 2 minutes to about 120 minutes, preferably about 5 minutes to about 30 minutes. Due to the oxalic acid reaction, the hemicellulose and some quantity of cellulose hydrolyses to respective sugars and rest of the cellulose and lignin and other solid impurities remains in the cake.

The supernatant is separated, by centrifugation or any filtration method to remove hemicellulose supernatant having pentose sugar further processed for fermentation step by a yeast preferably Pichia stipits. The remaining solids are mixed with water or evaporated condensate or recycled process stream to achieve aqueous slurry of the said solids.

### Alkaline Treatment

The slurry containing about 6% to about 25% by weight solids obtained after organic acid treatment is then subjected to a treatment with a mixture of water and about 0.5 to about 2% alkali preferably Sodium hydroxide, at a temperature range of about 80° C. to about 210° C. for a period of about 5 minutes to 360 minutes. The alkali treatment completely dissolves the lignin part and cellulose and hemicelluloses part remain in cake. The dissolved lignin part is separated and washed with water and sent for evaporation. The filter cake consisting of rich cellulose is reslurried using water or evaporated condensate and then subjected for cooling at about 30° C. to about 65° C. and pH is adjusted to about 8 to about 12 maintaining the solids at about 10% to about 20% by weight prior to enzymatic hydrolysis.

### Enzymatic Hydrolysis

The cellulose rich slurry is treated with more than one cellulytic enzyme capable of hydrolyzing the cellulose such as cellulose, beta glycosidase, endoglucanase and cellobiohydrolase with suitable filter paper unit, for a period of about 4 hours to about 30 hours, preferably about 4 hours to 24 hours for complete hydrolysis of cellulose. The temperature is kept at about 30° C. to about 60° C. preferably at about 50° C. to about 60° C. more preferably about 55° C. and the pH of the slurry is maintained in the range of about 4 to about 6 preferably in the range of about 4.8 to about 5.2. In the enzymatic treatment, cellulolytic enzymes are used which consist of a catalytic domain, a linking peptide and a binding domain. The oxalic acid has the potential as catalytic domain for constructing organic macromolecules for use in cellulose hydrolysis that mimic the action of enzymes. The tested domains consist of oxalic acid is dicarboxylic acid with dielectric constant 1.23 and 4.21 Pkas. The results show that acid catalyzed hydrolysis is proportional to H+ ion concentration. The tested oxalic acid catalyzes the less degradation of glucose and xylose as compared to sulfuric acid. Overall yield of glucose and xylose from cellulose and hemicelluloses are higher for the oxalic acid as compared to other treatments.

### Fermentation

The enzymatic hydrolysate is cooled to ambient temperature and then subjected to fermentation with suitable microorganism capable of producing ethanol from pentose and hexose sugars. Preferably, the fermenting microorganism is yeast of the genera Pichia and Saccharomyces. The microorganism used for pentose sugar utilization is Pichia stipitis and for hexose fermentation is Saccharomyces cerevisae in either active dry form or by using slant via propagation. The incubation time of the fermentation is about 12 hours to about 72 hrs preferably about 18 to about 72 hours and more preferably about 24 to about 48 hours. Fermentation is carried out at temperature about 25° C. to about 40° C. preferably at about 30° C. to 35° C. and more preferably at 31° C. to about 33° C. The pH during fermentation is about 4.5 to about 5.0. The fermentation is supplemented with proper dosing of nitrogen, micro and macro nutrients at a concentration sufficient to enhance the growth of microorganism.

### Distillation and Dehydration:

The fermented wash containing about 2% to about 7% alcohol is distilled and dehydrated to get about 99.8% fuel grade Ethanol. The spent mash or vinnase from the distillation is decanted/centrifuged/screw pressed to remove the water and the wet cake containing about 40% to about 55% solids is sent to boiler for the generation of steam and power.

### Evaporation of Thin Slops/Waste Water Streams:

The water washing streams are collected and subjected for 4 to 5 steps. The effect of evaporation is to recover the water. The condensate or water is recycled back in to the process.

This method further has advantage of obtaining less lignin derived by-product. The sugars obtained are of high purity and high yield. The lignin which is obtained is in reactive form as a powder and optionally other valuable by-products are obtained.

### EXAMPLES

The invention will now be illustrated with working examples. It is to be understood that the working examples illustrate the working of the process of the invention and are not intended to be taken restrictively to imply any limitation on the scope of the present invention. In the examples given, the feed stocks used are Sugar Cane or Sweet sorghum Bagasse, Corn Stover, Corn Cob, Grass, Distillers Dried Grains and Soluble (DDGS), and Distillers Wet Grains (DWG)

### Example-I

### Sugar Cane Bagasse as Feed Stock

10 kilograms of Cane Bagasse having moisture content in the range of about 5% to about 10% is subjected to mechanical action for size reduction to about 2 mm to about 20 mm having solid content of about 85% to about 95% by weight followed by addition of about 9234 grams of fresh water or recycled condensate from the evaporation step to the bagasse pieces so as to obtain a slurry. The total solids content in the slurry is about 6.0% by weight. The slurry is mixed with about 100 grams of oxalic acid was such that the concentration of organic acid present in the slurry is about 0.1% to about 2.0% by weight. The mixing is carried out in combination with heating at a temperature about 150° C. for a period of about 45 minutes. After the oxalic acid treatment, the slurry was subjected to filtration/centrifugation for the separation of the hemicellulose rich supernatant which is further fed for fermentation. The cake left after filtration is reslurried by using recycled water or fresh water to maintain dry solid concentration in the range of about 10% to about 20% by weight. The aqueous slurry is treated with a mixture of water and about 0.5% alkali at a temperature of about 150° C. for a period of about 30 minutes. The alkali treated slurry was cooled to about 40° C. and the pH was adjusted at about 4 followed by addition of cellulytic enzyme such as a combination of cellulase and beta Glucosidasae for a period of about 16 hours. The enzymatic hydrolysate is further subjected to fermentation for a period of about 24 hours at a temperature of about 31° C. to about 33° C. The microorganism used for pentose sugar was Pichia stipitis and for hexose was Saccharomyces cerevisae. The yeast addition is about 1.0 grams to about 1.5 grams per liter of fermentation media. The fermented wash containing about 4.4 by volume alcohol was distilled.

### Example-II

### Using Grass as Feed Stock

10 kilograms of grass having moisture content of about 50% to about 60% by weight is subjected to a mechanical action for size reduction to about 2 mm to 20 mm using shredder having solid contents in the range of about 40% to about 50%, followed by addition of about 5000 grams of fresh water or recycled condensate from the evaporation step so as to obtain slurry. The total solids content in the slurry obtained is about 6.0% by weight. The slurry is mixed with about 100 grams of oxalic acid such that the concentration of organic acid present in the slurry is about 0.1 to about 2.0% by weight. The mixing is carried out in combination with heating at a temperature about 130° C. for a period of about 60 minutes. After the oxalic acid treatment, the slurry was subjected to filtration/centrifugation for the separation of the hemicellulose rich supernatant which is further fed for fermentation. The cake left after filtration is reslurried by using recycled water or fresh water to maintain dry solid concentration in the range of about 10% to about 20% by weight. The aqueous slurry is treated with a mixture of water and about 1% alkali at a temperature of about 180° C. for a period of about 60 minutes. The alkali treated slurry was cooled to about 30° C. and the pH was adjusted at about 5 followed by addition of cellulytic enzyme such as a combination of cellulase and beta Glucosidasae for a period of about 16 hours. The enzymatic hydrolyasate is further subjected to fermentation for a period of about 48 hours at a temperature of about 31° C. to about 33° C. The microorganism used for pentose sugar was Pichia stipitis and for hexose was Saccharomyces cerevisae. The fermented wash containing about 3.8 by volume alcohol was distilled. It is observed that the hemicellulose pretreatment efficiency is about 50% to about 85% and the fermentation efficiency is in the range of about 70% to 85%.

### Example-III

### Using Distillers Wet Grains (DWG) as Feedstock

Distillers wet grains (DWG) is byproduct of grain like corn, wheat fermentation. The total solid content for DWG taken up for the process of present invention was in the range of about 30 to about 35% by weight, more preferably about 32% by weight. The moisture content was in the range of about 35% to about 40% by weight, more preferably about 38% by weight. 10 kg of DWG was pretreated. The total solids content was about 16% by weight. The organic acid used was in the range of about 0.5% to about 3.0% by weight. Temperature was maintained in the range of about 130° C. to about 170° C. for a period of about 60 minutes to 120 minutes. The enzymatic biochemical conversion was carried out at temperature of about 50° C. to about 60° C., more preferably at about 55° C. with pH maintained preferably at about 5.5. The enzymes used were Cellulase and Beta Glucosidase for a period of about 16 hours to about 24 hours, more preferably for about 16 hours. The fermentation of DWG was carried out at a temperature of about 30° C. to about 35° C., more preferably at about 31° C. to about 33° C. for a period of about 20 to about 50 hours, more preferably for about 24 hours to about 48 hours in presence of the yeast of about 1.0 grams to about 1.5 grams per liter of fermentation broth.

It is observed that the hemicellulose pretreatment efficiency is about 25% to about 45% and the fermentation efficiency is in the range of about 70% to 90%. The alcohol efficiency was in the range of 25% to 90%.

### Example-IV

### Using Corn Stover as Feedstock

10 kilogram of corn stover having moisture content of about 2% to about 8% more preferably about 7% by weight is subjected to a pretreatment having solid contents in the range of about 90% to about 95%, preferably about 95% by weight followed by addition of fresh water or recycled condensate from the evaporation step so as to obtain slurry. The total solids content in the slurry obtained is about 7.0% by weight. The slurry is mixed with about 100 grams of oxalic acid such that the concentration of organic acid present in the slurry is about 0.5% by weight. The mixing is carried out in combination with heating at a temperature about 170° C. for a period of about 60 minutes. After the oxalic acid treatment, the slurry was subjected to filtration/centrifugation for the separation of the hemicellulose rich supernatant which is further fed for fermentation. The cake left after filtration is reslurried by using recycled water or fresh water to maintain dry solid concentration in the range of about 10% to about 20% by weight. The aqueous slurry is treated with a mixture of water and about 1% alkali at a temperature of about 180° C. for a period of about 60 minutes. The alkali treated slurry was cooled to about 30° C. and the pH was adjusted at about 5 followed by addition of cellulytic enzyme such as a combination of cellulase and beta Glucosidasae for a period of about 16 hours. The enzymatic hydrolyasate is further subjected to fermentation for a period of about 48 hours at a temperature of about 31° C. to about 33° C. The microorganism used for pentose sugar was Pichia stipitis and for hexose was Saccharomyces cerevisae. The fermented wash containing about 4.4 by volume alcohol was distilled. It is observed that the hemicellulose pretreatment efficiency is about 55% to about 80% and the fermentation efficiency is in the range of about 50% to 85%.

### Example-V

### Using Corn Cob as Feedstock

Corn cob is a waste from corn plants consisting of remnants after the removal of corn grains and is available in the agricultural farms. Corn Cob having specific moisture is subjected to a pretreatment by addition of fresh water or recycled condensate from the evaporation step so as to obtain slurry. The slurry is mixed with about 200 grams of oxalic acid such that the concentration of organic acid present in the slurry is about 1% by weight. The mixing is carried out in combination with heating at a temperature about 130° C. for a period of about 30 minutes. After the oxalic acid treatment, the slurry was subjected to filtration/centrifugation for the separation of the hemicellulose rich supernatant which is further fed for fermentation. The cake left after filtration is reslurried by using recycled water or fresh water to maintain dry solid concentration in the range of about 10% to about 20% by weight. The aqueous slurry is treated with a mixture of water and about 1% alkali at a temperature of about 140° C. for a period of about 60 minutes. The alkali treated slurry was cooled to about 30° C. and the pH was adjusted at about 5 followed by addition of cellulytic enzyme such as a combination of cellulase and beta Glucosidasae for a period of about 16 hours. The enzymatic hydrolyasate is further subjected to fermentation for a period of about 48 hours at a temperature of about 31° C. to about 33° C. The microorganism used for pentose sugar was Pichia stipitis and for hexose was Saccharomyces cerevisae. The fermented wash containing about 4.4 by volume alcohol was distilled. It is observed that the hemicellulose pretreatment efficiency is about 55% to about 80% and the fermentation efficiency is in the range of about 80%. The enzymatic efficiency is in the range of about 75% to about 85%.

### Example-VI

### Using Distillers Dry Grains and Solubles (DDGS) as Feedstock

The total solid content for DDGS taken up for the process of present invention was in the range of about 90% to about 95% by weight, more preferably about 92% by weight. The moisture content was in the range of about 5% to about 10% by weight, more preferably about 8% by weight. The total initial solids content was about 20% to about 25% by weight. The organic acid treatment is carried out at a temperature was of about 130° C. for a period of about 30 minutes. The slurry was cooled at about 30° C. and the pH was adjusted to about 5. The slurry is subjected to enzymatic treatment with enzymes such as Cellulase and Beta Glucosidase for a period of about 16 hours. The fermentation of DDGS was carried out at a temperature of about 31° C. to about 33° C. for a period of about 48 hours in presence of the yeast of about 1.5 grams per liter of fermentation broth. It is observed that the hemicellulose pretreatment efficiency is about 55% to about 80% and the fermentation efficiency is about 80%. The alcohol efficiency was in the range of 25% to 90%.

It is to be noted that the present invention is susceptible to modifications, equivalent changes, adaptations, by those skilled in the art. Such modifications, equivalent changes, adaptations are within the scope of the present invention which is further set forth under the claims as follows.

1. A process for production of ethanol from lignocellulosic material comprising the steps of:
(a) subjecting the lignocellulosic material comprising hemicellulose, lignin and cellulose to a mechanical action reducing it to a finely divided form;
(b) mixing said reduced material with water or evaporated condensate or recycled process stream, producing aqueous slurry;
(c) reacting the aqueous slurry with an organic acid, preferably oxalic acid in combination with heating;
(d) separating the hemicellulose rich supernatant, solid wet cake from the slurry of step (c), subjecting said supernatant to pentose sugar fermentation;
(e) treating the solid wet cake of step (d) with water or evaporated condensate or recycled process stream, resulting an aqueous slurry, comprising insoluble lignin and cellulosic solids;
(f) reacting the slurry of step (e) with alkali, solubilizing the lignin; followed by cooling;
(g) separating the lignin soluble fraction, solid wet cake comprising cellulose from the slurry of step (f);
(h) washing the solid wet cake of cellulose of step (g) with fresh water or evaporated condensate or recycle process stream, producing aqueous slurry comprising of cellulose solids;
(i) adding at least one enzyme capable of digesting cellulose contained in the lignocellulosic material into glucose to aqueous slurry comprising of cellulose solids;
(j) fermenting the medium resulting from enzymatic hydrolysis of step (i), comprising adding an ethanol producing yeast;
(k) separating of ethanol by distillation;

2. A process as claimed in claim 1, wherein the lignocellulosic material is reduced to a size of about 2 mm to about 20 mm;

3. A process as claimed in claim 1, wherein the aqueous slurry comprising the reduced material, containing solids of about 5% to about 25% by weight;

4. A process as claimed in claim 1, wherein reacting the aqueous slurry with organic acid of about 0.1% to about 3% by weight, maintaining the heating at about 110° C. to about 210° C. for a period of about 2 minutes to about 120 minutes, having pH of about 4 to about 5.5;

5. A process as claimed in claim 1, wherein separating the hemicellulose rich supernatant and solid wet cake by a solid-liquid separation technique, preferably by means of filtration or centrifugation;

6. A process as claimed in claim 1, wherein feeding the hemicellulose rich supernatant to pentose sugar fermentation using a yeast, preferably Pichia stipitis, reslurrying the solid wet cake with water or evaporated condensate or recycled process stream;

7. A process as claimed in claim 1, wherein reacting the said aqueous slurry with alkali of about 0.5% to about 2% by weight, maintaining the temperature at about 80° C. to about 210° C., for a period of about 5 minutes to about 360 minutes, having pH of about 7 to about 9;

8. A process as claimed in claim 1 wherein cooling the alkali treated slurry at about 30° C. to about 65° C., followed by adjusting the pH at about 4.8 to about 5.2;

9. A process as claimed in claim 1, wherein separating the soluble lignin rich supernatant and solid wet cake comprising cellulose by a solid-liquid separation technique, preferably by means of filtration or centrifugation;

10. A process as claimed in claim 1, wherein treating the said cellulose containing slurry with one or more cellulytic enzyme selected from the group of cellulose, endoglucanase, cellobiohydrolase and beta-glycosidase maintaining the temperature at about 30° C. to about 60° C., for a period of about 4 hours to about 30 hours, at a pH of about 4.8 to about 5.2;

11. A process as claimed in claim 1, wherein fermenting the cooled media resulted from enzymatic hydrolysis with Saccharomyces cerevisae, maintaining the temperature at about 25° C. to 40° C., for a period of about 18 hour to about 72 hour; obtaining about 2.0% to about 7.0% by volume of ethanol;

12. a process as claimed in claim 1, wherein distilling the fermented broth followed by purification by dehydration yielding ethanol.
